(19) United States
(12) Patent Application Publication
(10) Pub. No.: US 2011/0223629 A1
(43) Pub. Date: Sep. 15, 2011

(54) METHOD FOR DETERMINATION OF DELTA-D VALUES OF NON- EXCHANGEABLE HYDROGEN STABLE ISOTOPES ON ETHANOL' S METHYL GROUP BY MEANS OF IRMS INSTRUMENTAL TECHNIQUE

(75) Inventors: Milanka Glavanovic, Pancevo (RS); Ivan Smajlovic, Becej (RS)

(73) Assignee: Ivan Smajlovic, Becej, RS (RS)

(21) Appl. No.: 13/119,238

(22) PCT Filed: Oct. 5, 2009
(86) PCT No.: PCT/RS2009/000023

§ 371 Date: Mar. 16, 2011

(30) Foreign Application Priority Data
Oct. 6, 2008 (RS) P-2008/0462

(51) Int. Cl. C12Q 1/32

(52) U.S. Cl. 435/26

(57) ABSTRACT

The present innovation is related with chemical instrumental analysis, i.e. to the domain of analysis of stable isotopes in food products, and it is related to the method for preparation of ethanol samples and mode for determination of isotopic ratio of non-exchangeable hydrogen stable isotopes sited on the methyl site of ethanol by means of instrumental technique CF-TC/EA-IRMS (Continuous Flow-Temperature Conversion/Elemental Analyzer-Isotope Ratio Mass Spectrometry), and for the purpose of establishing authenticity and geographical origin of wine and grape must, beer, alcoholic beverages, fruit juices, honey, vinegars and other food products which contain alcohol and/or fermentable sugars. This method is based on full enzymatic (or organic) transformation of ethanol samples to gain ethanoic acid (acetic acid), controlled neutralization of acetic acid and further concentration, purification of prepared acetate salt and its drying to the constant mass and further determination of δD values in prepared samples by means of CF-TC/EA-IRMS.

### TECHNICAL FIELD

The present innovation is related with chemical instrumental analysis, i.e. to the domain of analysis of stable isotopes in food products, and it is related to the process for preparation of ethanol samples and mode for determination of isotopic ratio of non-exchangeable hydrogen stable isotopes sited on the methyl site of ethanol by means of instrumental technique CF-TC/EA-IRMS (Continuous Flow-Temperature Conversion/Elemental Analyzer-Isotope Ratio Mass Spectrometry), and for the purpose of establishing authenticity and geographical origin of wine and grape must, beer, alcoholic beverages, fruit juices, honey, vinegars and other food products which contain alcohol and/or fermentable sugars.

### BACKGROUND ART

Isotopic methods have shown that they can be very powerful analytical tool for authenticity and geographical origin determination of wines and strong spirits. By measuring the content of stable isotopes in these products useful information can be provided for detection of many frauds in wine and strong spirits production. Instrumental techniques which are used for isotopic measurements are based on measuring the relative ratios of stable isotopes by means of Isotope Ratio Mass Spectrometry.

Systems comprising a pyrolysis chamber and continuous flow isotope spectrometer CF-TC/EA-IRMS (Flash HT Continuous Flow-Temperature Conversion/Elemental Analyzer-Isotope Ratio Mass Spectrometry) are commercially available for stable hydrogen analysis of solid and liquid samples.

When analyzing ethanol samples, by means of CF-TC/EA-IRMS (Continuous Flow-Thermal Conversion/Elemental Analyzer-Isotope Ratio Mass Spectrometry), because of the ethanol's hydroxyl group, which contains easily exchangeable hydrogen, gained δD values for ethanol of the same botanical and geographical origin can vary, and for that reason it is impossible to perform qualitative and quantitative identification of the ethanol sample origin.

One of the problems which can occur, for example, in strong spirit production, is in finalization production steps. The distillate is diluted with water to determined alcoholic strength which is necessary so that alcoholic drink could be consumed. By adding water which has different isotopic content, dynamic isotopic equilibrium is disturbed and hydrogen or deuterium which is bonded to oxygen atom of the hydroxyl group is exchanged. This is one of the reasons for gaining the wrong δD values and wrong information about ethanol origin.

Because of the problems which are stated above, instrumental technique CF-TC/EA-IRMS can not be very useful in detection of frauds in wine and alcoholic drinks production and moreover for detection of ethanol which originates from beet sugar, barley, wheat etc. in wine and alcoholic drinks.

According to the first aspect of this invention, the process for preparation of ethanol samples and mode for the determination of isotopic ratio of non-exchangeable hydrogen stable isotopes sited on the methyl site of ethanol by means of instrumental technique CF-TC/EA-IRMS (Continuous Flow-Temperature Conversion/Elemental Analyzer-Isotope Ratio Mass Spectrometry) is based on full enzymatic (or organic) transformation of ethanol samples to gain ethanoic acid (acetic acid), controlled neutralization of acetic acid and further concentration, purification of prepared acetate salt and its drying to the constant mass and further determination of δD values in prepared samples by means of CF-TC/EA-IRMS.

### BACKGROUND ART

Deuterium and hydrogen relative ratio measurements for the proposal of authenticity and origin determination of wines and alcoholic drinks, beer, fruit juices and honey today is done by means of NMR methodology (Site Natural Isotopic Fractionation-Nuclear Magnetic Resonance) which is based on intermolecular scanning of the measured ethanol sample and determination of isotopic composition of hydrogen and deuterium atoms sited on the first and on the second carbon atom inside of the ethanol molecule. Results gained by NMR methodology give the information about the presence of ethanol which originates from beet sugar or other industrial plants, and which belongs to the C3 plant group.

This instrumental technique has limited use in practice, because it can be used only if database for wine samples is previously made and that without specific database conclusions can be very subjective and quantitative analysis hard. For that reason it is used only for wine with designated geographical origin for which databases are previously made. Other lacks of this instrumental technique are:
NMR methodology is very time consuming method
It requires big consumption of helium and liquid nitrogen and also electrical energy,
It occupies big part of the working place because of it's size and because of the very strong magnetic field which it makes (security zone is needed),

### DISCLOSURE OF INVENTION

The main goal of this invention is to overcome the barrier and lacks of today known apparatus and methods for determination of isotopic composition of non-exchangeable hydrogen and deuterium atoms in ethanol samples.
According to the first aspect of this invention, the method for preparation of ethanol samples and mode for the determination of isotopic ratio of non-exchangeable hydrogen stable isotopes sited on the methyl site of ethanol (CH₃— group) is based on the full enzymatic transformation of ethanol samples to acetic acid (ethanoic acid) in presence of enzymes ethanol-dehydrogenase and acetaldehyde-dehydrogenase and coenzyme NAD⁺, controlled neutralization of gained acetic acid to make acetate, purification of prepared acetate and vaporization to the constant mass and further determination of δD values in prepared samples by means of CF-TC/EA-IRMS.

According to a further aspect of this invention, conversion of ethanol into acetic acid alternatively can be done by organic oxidation of ethanol in presence of sodium-dichromate (IV).

Also according to a further aspect of this invention, method for preparation of ethanol samples and their conversion into acetic acid alternatively can be done by recirculation previously isolated ethanol sample through the continuous flow column with mixture of two immobilized enzymes ethanol-dehydrogenase and acetaldehyde-dehydrogenase and in presence of coenzyme NAD⁺.

### BEST MODES FOR CARRYING OUT OF THE INVENTION

According to the first aspect of this invention, the method for preparation of ethanol samples and mode for the determination of isotopic ratio of non-exchangeable hydrogen stable isotopes sited on the methyl site of ethanol (CH₃— group) is based on the full enzymatic transformation of ethanol samples to acetic acid (ethanoic acid) in presence of enzymes ethanol-dehydrogenase and acetaldehyde-dehydrogenase and coenzyme NAD⁺, controlled neutralization of gained acetic acid to make acetate, purification of prepared acetate and vaporization to the constant mass and further determination of δD values in prepared samples by means of CF-TC/EA-IRMS.
First step of the method is based on the isolation of ethanol from wine, strong spirits, fermented fruit juices fermented honey solutions samples by distillation. Prior to distillation it is needed to neutralize all volatile acids in the sample which could past over into the distillate.
Second step is based on the full enzymatic transformation of previously isolated ethanol to produce acetic acid without isotopic fractionation. This is done in presence of two specific enzymes, ethanol dehydrogenase and acetaldehyde dehydrogenase and oxido-reduction coenzyme NAD⁺.
Third step involves isolation of gained acetic acid from the reaction mixture without any quantitative lost by distillation in presence of overheated water vapors or by vacuum distillation.
Forth step involves neutralization of isolated acetic acid with Sodium Hydroxide or some other alkali base, Sodium Carbonate or similar, in presence of pH-meter until pH value of 8.1 and in that way preparation of acetate salt (preferable Sodium Acetate).
Step five involves concentrating by means of vacuum distillation until the solution gets syrupy consistence. The distillate can be rejected.
Sixth step involves purification of cooled Sodium Acetate with diethyl ether to remove all organic impurities which are lapsed n the matrix. It can be by using ultrasonic water bath and then removal of the ether phase with help of the separation glass. This step should be repeated three times. After purification vaporization of Sodium acetate can be done to dry and constant mass.

Further step involves determination of the relative ratio of hydrogen stable isotopes in prepared Sodium Acetate sample which comes from un-exchanged methyl group inside of acetate salt.

According to a further aspect of this invention, at the beginning of the method, prior to distillation and isolation of ethanol from wine, strong spirits, fermented fruit juices or fermented honey solutions samples, they must be neutralized to avoid isotopic fractionation and further loss during the distillation step by addition of sodium hydroxide solution (NaOH) until pH value reach 8.1 to 8.5. It that way all present organic acids will be neutralized and present carbonyl compounds-aldehydes and ketons will be retrogressed. In this way after distillation, gained distillate is acids free and further process steps are the same as described prior in the text.

According to a further aspect of this invention, second step of the method which involves ethanol conversion to acetic acid alternatively can be carried out by means of organic oxidation of ethanol in presence of Sodium dichromate (IV) to gain acetic acid.

Also, according to a further aspect of this invention second step of the method which involves ethanol conversion to acetic acid also can be alternatively be carried out by recirculation of previously isolated neutralized ethanol from analyzed samples, in presence of NAD⁺ trough the column with immobilized mixture of ethanol-dehydrogenase and acetaldehyde dehydrogenase enzymes and for the purpose of faster biochemical conversion of ethanol into acetic acid.

In accordance with the idea of this invention, the method for preparation of ethanol samples and mode for the determination of isotopic ratio of non-exchangeable hydrogen stable isotopes sited on the methyl site of ethanol (CH₃— group), and for the purpose of authenticity and geographical origin determination of wines and grape musts, beers, alcoholic drinks, fruit juices, honey and all other food products which contain alcohol and/or fermentable sugars, it has it's advantages:
In the first place, it gives very good precision and repeatability of results for δD values of analyzed ethanol samples, no matter if ethanol sample was diluted with water before distillation, and it gives espied constant difference and dependence between ethanol samples with botanical origin from C3 group of plants;
There is no need for big financial funds and special conditions for maintenance like which is the case with instrumental technique NMR methodology,
It gives the opportunity to detect the presence of ethanol which originates from beet sugar, wheat, barley and other industrial plants which belong to the C3 group of plants, in the ethanol samples which are isolated from the analyzed wines and alcoholic drinks or fermented juices and fermented honey.

It will be appreciated that modifications to the embodiments described above are of course possible. Accordingly the present innovation is not limited to the embodiments described above.

### INDUSTRIAL APPLICABILITY

The method for preparation of ethanol samples and mode for the determination of isotopic ratio of non-exchangeable hydrogen stable isotopes sited on the methyl site of ethanol (CH₃— group) is applicable in instrumental analytical chemistry and is used for authenticity and geographical origin determination of wines and grape musts, alcoholic drinks, beers, fruit juices, honey and other food products which contain ethanol and/or fermentable sugars.

1-5. (canceled)

6. A process for determining origin of an alcohol-containing food product, comprising:
providing a small-quantity sample of the alcohol-containing food product; neutralizing volatile acids in the provided sample;
degrading carbonyl compounds: aldehydes and ketones in the provided sample; extracting an ethanol sample from the provided sample;
providing enzymes ethanol dehydrogenase and acetaldehyde dehydrogenase, and a oxido-reduction coenzyme NAD⁺ to convert ethanol in the extracted ethanol sample to acetic acid;
extracting the acetic acid, wherein the step of extracting the acetic acid includes at least one of the following processes: distillation in the presence of overheated water vapor, or vacuum distillation;
producing an acetate salt solution from the extracted acetic acid by neutralizing the extracted acetic acid until the pH value of solution reaches 8.1;
concentrating the produced acetate salt solution by vacuum distillation until the acetate salt reaches syrupy consistency, wherein a byproduct distillate is discarded; cooling the concentrated acetate salt solution;
removing impurities from the concentrated and cooled acetate salt solution by adding an organic solvent and mixing the solution in an ultrasonic water bath, wherein the organic solvent dissolves the impurities;
discarding the organic solvent containing the dissolved impurities using a separation glass;
solidifying the acetate salt by removing water from the purified concentrated acetate salt solution;
measuring an isotopic composition, i.e., isotopic relative ratio of deuterium and hydrogen of the solidified acetate salt;
calculating a δD value for the measured isotopic composition; and
comparing the calculated δD value with δD values associated with a food product with known origins.

7. The process of claim 6, wherein the step of neutralizing volatile acids includes adding Sodium Hydroxide solution until pH value of the mixture reaches value between approximately 8.1 and 8.5.

8. The process of claim 6, wherein the step of producing an acetate salt solution from the extracted acetic acid by neutralizing the extracted acetic acid until the pH value of solution reaches 8.1, includes using an alkaline base.

9. The process of claim 8, wherein the alkaline base is Sodium Hydroxide.

10. The process of claim 6, wherein the step of producing an acetate salt solution from the extracted acetic acid by neutralizing the extracted acetic acid until the pH value of solution reaches 8.1, includes using a weak acid salt.

11. The process of claim 10, wherein the weak acid salt is Sodium Carbonate.

12. The process of claim 6, wherein the organic solvent used in the steps of removing impurities from the concentrated and cooled acetate salt solution, and discarding the organic solvent containing the dissolved impurities, includes a diethyl ether.

13. The process of claim 6, wherein the steps of removing impurities from the concentrated and cooled acetate salt solution, and discarding the organic solvent containing the dissolved impurities, are repeated at least three times.

14. The process of claim 6, further comprising:
providing a column with an immobilized mixture of ethanol dehydrogenase enzyme and acetaldehyde dehydrogenase enzymes;
adding oxido-reduction coenzyme NAD⁺ to the extracted ethanol sample; and
recirculating the mixture of the ethanol sample and the oxido-reduction coenzyme NAD⁺ through the provided column.
